(19) United States
(12) Patent Application Publication
(10) Pub. No.: US 2007/0265659 A1
(43) Pub. Date: Nov. 15, 2007

(54) Adhesive sutures

(75) Inventor: Warren P. Williamson IV, Loveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: CleveX, Inc.

(21) Appl. No.: 11/801,607

(22) Filed: May 10, 2007

Related U.S. Application Data
(60) Provisional application No. 60/799,088, filed on May 10, 2006.

(51) Int. Cl. A61B 17/06

(52) U.S. Cl. 606/224

(57) ABSTRACT

An apparatus for joining body tissues with adhesive sutures includes a suture needle has first and second longitudinally spaced needle ends, the first needle end having a needle point. A suture filament has first and second longitudinally spaced filament ends, the first filament end being attached to the second needle end. A filament sheath has first and second sheath ends and defines a tubular sheath void. At least a portion of the suture filament is enclosed within the sheath void. The first sheath end is attached to the second needle end. An adhesive source is connectable to the second filament end and the second sheath end. The adhesive source is adapted to selectively provide an adhesive to the sheath void. A method of using the apparatus is also provided.

### RELATED APPLICATION

This application claims priority from U.S. Provisional Application No. 60/799,088, filed May 10, 2006, the subject matter of which is incorporated herein by reference.

### TECHNICAL FIELD

The present invention relates to an apparatus and method for using sutures and, more particularly, for joining body tissues with adhesive sutures.

### BACKGROUND OF THE INVENTION

The art of suturing tissue has been known for a very long time. Many developments regarding sutures are directed toward the suture material or the deployment of the suture, such as various different knots or knot-tying configurations. There have been many ways disclosed in the prior art to use a tensile member (suture) without tying knots. The prior art also includes adjustable stop beads, crimpers, and plates; all of which rely on the surface of the skin to hold the tensile force required and approximate the edges of the wound for healing. Scar tissue forms at the anchor point of the bead, crimper, plate, or other stop device in response to the trauma of concentrating the forces at the penetration site.

When closing a wound in tissue the most important aspect is to approximate the wound edges, thus stopping the blood flow and bringing the tissue edges together to begin the healing process. A simple interrupted stitch is the most common way to do this. While effective at bringing the severed edges of the skin together, an interrupted stitch technique can leave a scar which resembles a railroad track. This is unsightly and undesirable by the patient in most cases. When the patient would like to have a skin lesion removed and have little or no scar remaining, they are often referred to a surgical dermatologist or a plastic surgeon. These specialists have been trained in special suturing techniques to reduce the scarring of the skin. When plastic surgeons are concerned with making the least visible scar, they perform a running suture technique which looks like a zigzag between each side of the skin. These sutures are very carefully placed just below the skin's surface. An additional set of sutures are placed lower in the wound to approximate the deeper tissues. This eliminates the penetration of the needle through the skin edges and eliminates the high stress point points created when pulling not tight above the surface of the skin. However, such careful placement, tensioning, and knot-tying can be very time-consuming.

An example of an alternate suture structure is disclosed in U.S. Pat. No. 6,264,675, issued Jul. 24, 2001 to Brotz (hereafter referenced as the '675 patent). The '675 patent discloses a suture for closing two sides of an incision or cut in human skin or other body tissue, and more particularly relates to a suture having adhesive thereon (col. 1, lines 5-9). The suture is of a thread material having a needle member disposed at one end and adhesive disposed along the suture so that when the suture is drawn into body tissue, the adhesive becomes activated (col. 2, lines 15-19). In some embodiments, the adhesive can have a dissolving coating (col. 2, lines 42-43). The adhesive can be a mixture activated by solvation caused by moisture within the body tissue or the adhesive can be a plurality of alternate segments of adhesive which, when mixed by solvation and pulled through the body tissue, become activated (col. 2, lines 43-47). However, the suture of the '675 patent requires careful handling to avoid accidental activation of the adhesive through exposure to environmental moisture. Additionally, the adhesive may activate prematurely when the suture is placed in the body tissue, thus preventing the surgeon from adjusting the suture as desired. Finally, the adhesive coating and/or any dissolving coating over the adhesive may deteriorate over time during storage or after exposure to air, adversely effecting the operation of the suture.

Accordingly, it is desirable to provide a method and apparatus of a suture which: securely holds body tissues together, promotes healing, may be stably stored, withstands environmental moisture without activating, may be used in a timely and efficient manner, and is economical to manufacture and use.

### SUMMARY OF THE INVENTION

In an embodiment of the present invention, an apparatus for joining body tissues with adhesive sutures is described. A suture needle has first and second longitudinally spaced needle ends, the first needle end having a needle point. A suture filament has first and second longitudinally spaced filament ends, the first filament end being attached to the second needle end. A filament sheath has first and second sheath ends and defines a tubular sheath void. At least a portion of the suture filament is enclosed within the sheath void. The first sheath end is attached to the second needle end. An adhesive source is connectable to the second filament end and the second sheath end. The adhesive source is adapted to selectively provide an adhesive to the sheath void.

In an embodiment of the present invention, a method of joining body tissues using adhesive sutures is described. A suture apparatus including a suture needle having first and second longitudinally spaced needle ends, the first needle end having a needle point, and a suture filament having first and second longitudinally spaced filament ends, the first filament end attached to the second needle end, is provided. A filament sheath having first and second sheath ends and defining a tubular sheath void, at least a portion of the suture filament enclosed within the sheath void, the first sheath end adjacent the second needle end, is provided. First and second body tissues are approximated into a suture position with at least a portion of the suture apparatus extending therethrough. Adhesive is provided to at least a portion of the suture filament. At least a portion of the suture filament is retained within the first and second body tissues. The filament sheath is withdrawn from the first and second body tissues.

### BRIEF DESCRIPTION OF THE DRAWINGS

For a better understanding of the invention, reference may be made to the accompanying drawings, in which:

FIG. 1A is a side view of a first embodiment of the present invention;

FIG. 1B is a partial cross-sectional view taken along line 1B-1B in FIG. 1A;

FIGS. 2A-2D illustrate a sequence of operation of the embodiment of FIG. 1A;

FIG. 3 is a side view of a second embodiment of the present invention;

FIG. 4 is a side view of a third embodiment of the present invention;

FIG. 5A is a side view of a fourth embodiment of the present invention;

FIG. 5B is a partial cutaway view of an example component for use with any embodiment of the present invention; and

FIG. 6 is a partial cutaway view of another example component for use with any embodiment of the present invention.

### DESCRIPTION OF EMBODIMENTS

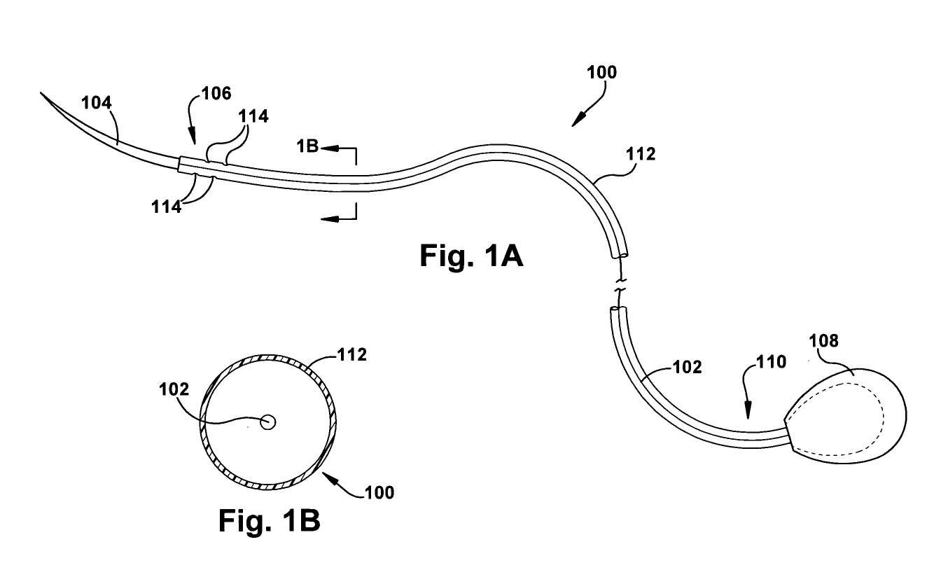

In accordance with a first embodiment of the present invention, FIGS. 1A and 1B depict an apparatus 100 for joining body tissue with adhesive sutures. The apparatus provides for a single use suture 102 with a needle 104 at the proximal end 106 and an ampule of adhesive or other adhesive source 108 at the distal end 110. The suture 102 may be absorbable or non-absorbable. The suture 102 is covered with a sheath 112 which is in fluid communication with the adhesive ampule 108. The suture 102 may be directly connected to the adhesive ampule 108 or may be connected through one or more other structures, such as the sheath 112. The sheath 112 may be, for example, a heat shrink tubing made by Advanced Polymers Incorporated, of Salem, N.H., and having a wall thickness of 0.00025″. One or more vent openings 114 may be provided in the sheath 112 near the proximal end 106.

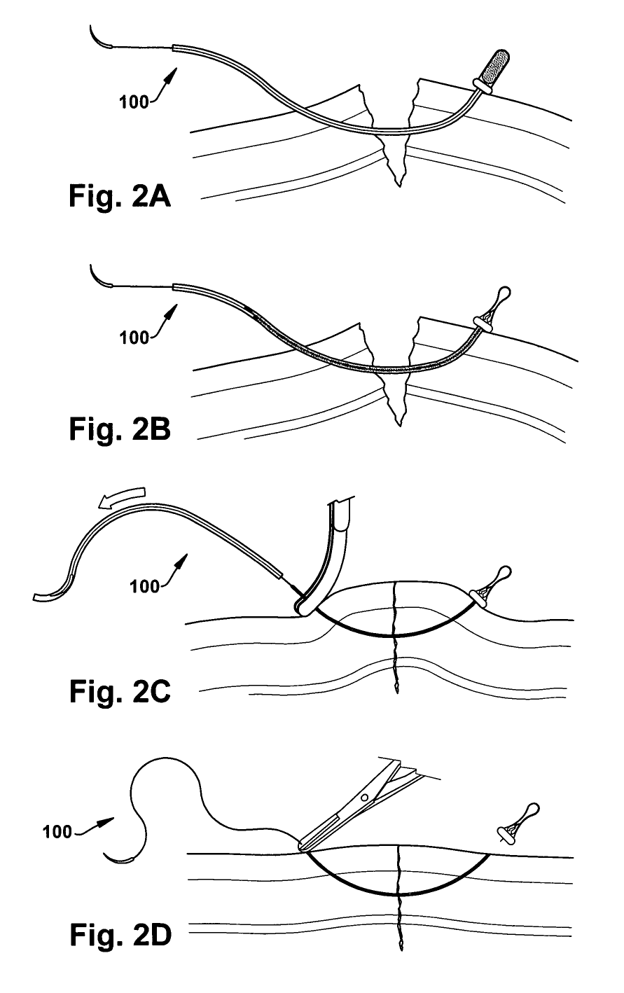

FIGS. 2A, 2B, 2C, and 2D illustrate a sequence of operation of the apparatus 100 according to the first embodiment. In FIG. 2A, the suture needle 104 is passed through the first side of the wound below the dermis, through the wound opening, and out through the dermis of the opposite side, similarly to the known technique of passing the suture 102 for an interrupted stitch. Once the suture 102 is placed, as shown in FIG. 2B, the ampule 108 is crushed, forcing liquid adhesive to wick down the suture 102 inside the sheath 112. The vent openings 114, when present, allow air to exhaust from inside the sheath 112 as the adhesive enters, and consequently draw the adhesive into the sheath 112. In FIG. 2C, the sheath 112 is pulled proximally toward the needle 104, thus exposing the adhesive-impregnated suture 102 to the wound. If the sheath 112 is sufficiently thin-walled, the material of the sheath will bunch up or telescope adjacent the needle 104 and exit the wound without requiring the needle to be moved. Tension is applied to the wound to approximate the edges of the tissue. The adhesive quickly sets up when exposed to the tissue and secures the suture 102 within the tissue.

This process is repeated across the wound line until it is closed. Once the wound is approximated, both ends of the suture 102 are tensioned and the ends are trimmed adjacent to the skin surface, as shown in FIG. 2D. The cut ends of the suture 102 will disappear below the surface of the skin as the edema resolves and the wound flattens. When the suture 102 is absorbable, no suture removal is needed and the suture will dissolve away for absorption by the body over time. This procedure is simple and straightforward and produces a knotless interrupted suture line.

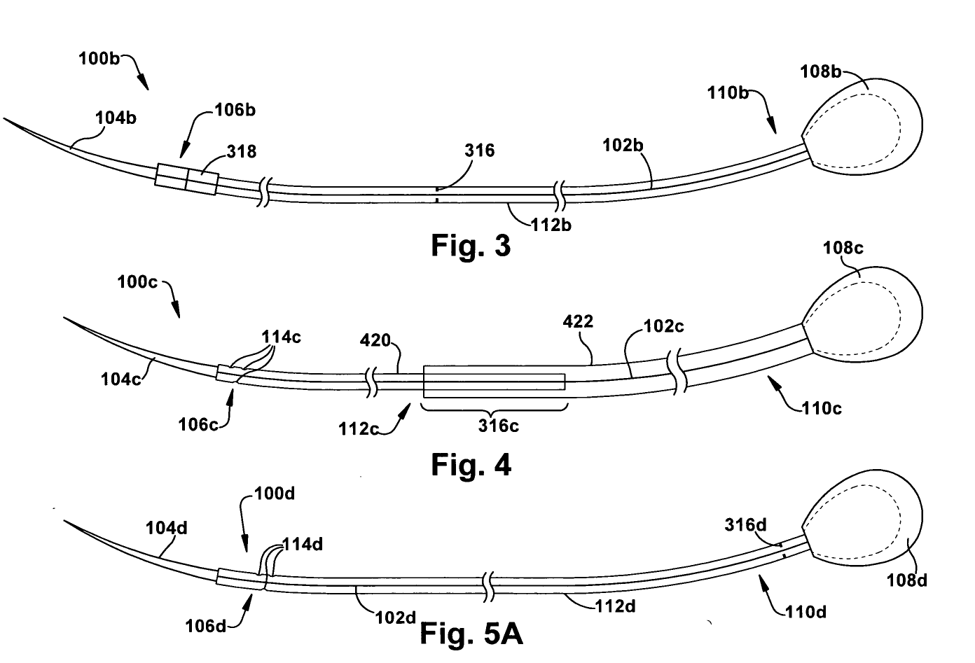

FIG. 3 depicts an apparatus 100b in accordance with a second embodiment of the present invention. Features of FIG. 3 that are the same as or similar to those described previously are given the same reference numbers with the addition of the suffix “b”. Description of common elements and operation similar to those in the previously described embodiment will not be repeated with respect to the second embodiment.

The apparatus 100b depicted in FIG. 3 includes at least one frangible area 316 spaced from both the proximal and distal ends 106 and 110 of the apparatus 100. The frangible area 316 may be a perforation pattern, scored pattern, reduced-thickness area, or other breakaway feature allowing the sheath 112 to split into two or more pieces or sheath fragments under an applied force.

When the frangible area 316 includes perforations of the sheath 112, the perforations may act as vent openings 114b, as described above. In such case, an additional adhesive source 318 may be provided between the frangible area 316 and the needle 104b. The adhesive source 318, when provided, acts to supply adhesive to a length of the suture 102b between the frangible area 316 and the adhesive source. Adhesive flowing from the adhesive ampule 108b may be diverted out of the sheath 112 through the perforations and, without the adhesive source 318, that portion of the suture 102b opposite the frangible area 316 from the adhesive ampule 108b may not be exposed to adhesive.

The operation of the apparatus 100b according to the second embodiment is similar to that previously described. However, the frangible area 316 may be positioned in the wound in a desired manner to supply adhesive directly to the wound surface. For example, if the frangible area 316 is positioned at or near the wound, instead of being buried in the surrounding skin, adhesive may flow out of the sheath 112b and directly into the wound, to help to secure the wound opening. The adhesive may flow out of the sheath 112b through perforations associated with the frangible area 316 and may be released from within the sheath when opposing tensile forces are applied to the sheath adjacent the proximal and distal ends 106b and 110b of the suture 102b to break the sheath apart.

FIG. 4 depicts an apparatus 100c in accordance with a third embodiment of the present invention. Features of FIG. 4 that are the same as or similar to those described previously are given the same reference numbers with the addition of the suffix “c”. Description of common elements and operation similar to those in the previously described embodiments will not be repeated with respect to the third embodiment.

The apparatus 100c according to the third embodiment is similar to the apparatus 100b according to the second embodiment in that the sheath 112c in both embodiments is arranged to part in a midsection thereof. The apparatus 100c, shown in FIG. 4, does so through a telescoping arrangement between a first sheath portion 420, surrounding a proximal portion 106c of the suture 102c, and a second sheath portion 422, surrounding a distal portion 110c of the suture. The relative sizing of the first and second sheath portions 420 and 422, to determine which of these at least partially surrounds the other, may be readily chosen by one of ordinary skill in the art for a particular application of the apparatus 100c.

The area of overlap between the first and second sheath portions 420 and 422 may be considered to constitute a frangible area 316c. Vent openings 114c may be provided to the apparatus 100c of the third embodiment to aid in wicking adhesive along the suture 102c from the adhesive ampule 108, particularly if the structure of the frangible area 316c is resistant to flow of air out of the sheath 112c.

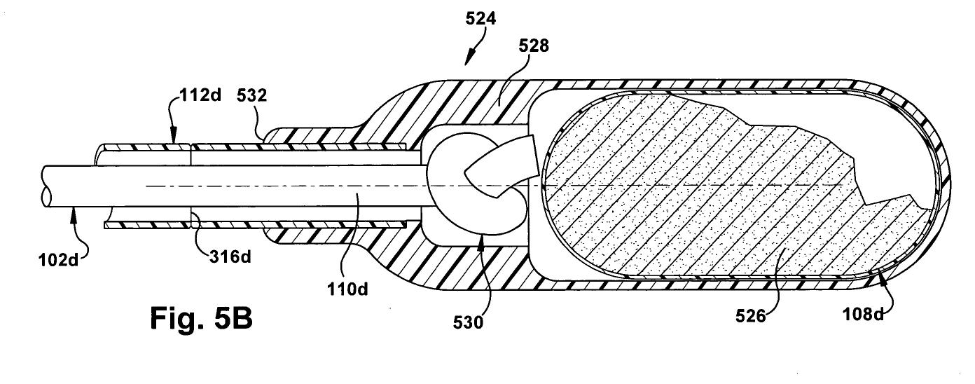

FIGS. 5A and 5B depict an apparatus 100d in accordance with a fourth embodiment of the present invention. Features of FIGS. 5A and 5B that are the same as or similar to those described previously are given the same reference numbers with the addition of the suffix “d”. Description of common elements and operation similar to those in the previously described embodiments will not be repeated with respect to the fourth embodiment.

The apparatus 100d according to the fourth embodiment is similar to the apparatus 100b and 100c according to the second and third embodiments in that the sheath 112d includes a frangible area 316d adapted to allow the sheath 112d to break away under force. The frangible area 316d of the apparatus 100d shown in FIGS. 5A and 5B, though, is located adjacent a distal end 110d of the suture 102d, to allow the sheath 112d to break away from the adhesive source 108. The suture 102d may be attached directly to the adhesive source 108d, or the suture may be connected to the adhesive source through a manifold structure 524, such as that shown in FIG. 5B.

The manifold structure 524 shown in FIG. 5B is described with reference to the apparatus 100d according to the fourth embodiment, but may be supplied to an apparatus 100 using any embodiment of the present invention. The manifold structure 524 may include an adhesive source 108d, such as a breakable ampule, containing a fluid adhesive 526. The adhesive source 108d is contained within a flexible cartridge 528. An anchor knot 534 or other stopper knot or stop bead structure (not shown) attaches a distal end of the suture 110d to the cartridge 528. The sheath 112d is attached to the cartridge 528 in fluid communication with the adhesive source 108d.

Optionally, and as shown in FIG. 5B, a frangible area 316d is located at or very near the manifold structure 524. When the frangible area 316d includes perforations, vent openings 114d may be provided in the sheath 112d at or near the proximal end 106d of the suture to encourage the adhesive to flow in a desired manner, rather than out through the perforations at the frangible area. Alternately, the frangible area 316d including perforations may be located at or just inside a mouth 532 of the manifold portion 524. Thus, the mouth 532 of the manifold portion 524 can at least partially block free access of the perforations to the surrounding atmosphere while still permitting the sheath 112d to separate at the frangible area 316d as desired.

Using the apparatus 100d according to the fourth embodiment, a surgeon may draw the needle, suture, and sheath 104d, 102d, and 112d through the patient's flesh, including the wound. The suture 102d may be drawn taut to approximate the wound while pulling the adhesive source 108d or manifold structure 524 into contact with the flesh at the entry point of the apparatus 100d into the patient. The adhesive source 108d or manifold structure 524 will act as a “stop” to assist in holding the wound together as the sheath 112d is broken at the frangible area 316d and pulled through the wound along the suture 102d.

When the sheath 112d is completely retracted through the patient's flesh toward the needle 104d, the suture 102d is exposed on a side of the wound opposite the adhesive source 108d. The suture 102d may then be cut and the apparatus 100d discarded.

The adhesive source 108d may be actuated at any suitable time relative to the separation and retraction of the sheath 112d. To actuate the adhesive source 108d shown in FIG. 5B, the user applies pressure to the manifold structure 524, which deforms sufficiently for the pressure to break a glass or other breakable ampule of adhesive 526 within the manifold structure 524. Adhesive 526 then flows past the anchor knot 530 and into the sheath 112d, possibly wicking down and through the material of the suture 102d during flow through the sheath 112d. The adhesive 526 is directed to the suture 102d by the sheath 112d, when the sheath is not retracted from the suture until after the adhesive source 108d is actuated. The adhesive 526 may squirt out into the wound in the fourth embodiment during retraction of the sheath 112d, possibly providing a desired adhesion within the wound.

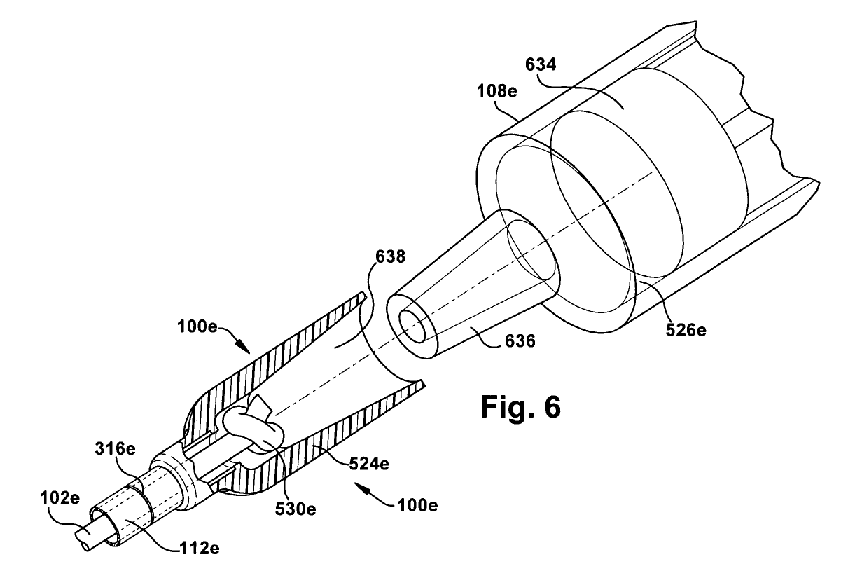

FIG. 6 depicts an apparatus 100e in accordance with a fifth embodiment of the present invention. Features of FIG. 6 that are the same as or similar to those described previously are given the same reference numbers with the addition of the suffix “e”. Description of common elements and operation similar to those in the previously described embodiments will not be repeated with respect to the fifth embodiment.

The adhesive source 108e shown in FIG. 6 is described with reference to the apparatus 100e according to the fifth embodiment, but may be supplied to an apparatus 100 using any embodiment of the present invention. The adhesive source 108e is selectively connectable to at least one of the suture 102 and the sheath 104 through a manifold structure 524e and is adapted to selectively provide an adhesive 526e to the sheath void 112e.

The adhesive source 108e may be a variable volume reservoir, such as the syringe shown in FIG. 6. The syringe 108e has a plunger 634 and contains adhesive 526e. The syringe 108e has a first fitting 636, which is adapted to selectively mate with a second fitting 638 of the manifold structure 524e. The first and second fittings 636 and 638 are optionally male and female luer lock fittings 636 and 638, respectively, as shown in FIG. 6 and described herein, but may be any suitably connecting structures, particularly those adapted for rapid connection and disconnection. When the luer lock fittings 636 and 638 are mated together, a fluid connection is formed between the syringe 108e and the suture 102e. This connection may be formed through or around the anchor knot 530e holding the suture 102e and manifold structure 524e together, as shown in FIG. 6. A frangible area 316e may be provided, as discussed above with reference to the second through fourth embodiments, to allow separation of the sheath 112e from the manifold structure 524e.

The apparatus 100e of the fifth embodiment operates similarly to that of at least the fourth embodiment, described above. However, the adhesive source 108e is actuated differently in the fifth embodiment. Namely, the syringe 108e may be connected to the manifold structure 524e as previously discussed. The user then applies pressure to the plunger 634 of the syringe 108e, or otherwise reduces the volume of the variable volume reservoir, to dispense a desired amount of adhesive 526e, which flows through the sheath 112e and along the suture 102e. The suture 102 is then cut and the apparatus 100e removed from the wound area as with any of the other embodiments of the present invention.

The use of a removable adhesive source 108e, such as a syringe, facilitates an “assembly line” style of activation of multiple apparatus 100e. For instance, the user may use several apparatus 100e to place a series of suture 102e stitches along a wound line, drawing each stitch taut to approximate the wound and adjusting each as needed. The user then can sequentially supply adhesive 526e to each of the apparatus 100e in turn from the same syringe 108e.

While aspects of the present invention have been particularly shown and described with reference to the preferred embodiment above, it will be understood by those of ordinary skill in the art that various additional embodiments may be contemplated without departing from the spirit and scope of the present invention. For example, the materials used in the apparatus 100 may differ as needed for particular applications. Multiple adhesive ampules or sources 108 or 318 may be provided to a single apparatus 100. The manifold structure 524 may have a different configuration than that described, while still facilitating a fluid connection between an adhesive source 108 and at least one of the suture 102 and the sheath 112. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of: securely holding body tissues together, promoting healing, being stably stored, withstanding environmental moisture without activating, being usable in a timely and efficient manner, and being economical to manufacture and use. Such advantages are particularly worthy of incorporating into the design, manufacture, and operation of adhesive sutures. In addition, the present invention may provide other advantages which have not yet been discovered.

Other aspects, objects, and advantages of the present invention can be obtained from a study of the drawings, the disclosure, and the appended claims.

Having described the invention, the following is claimed:

1. An apparatus for joining adjacent body tissues with adhesive sutures, the apparatus comprising:
a suture needle having first and second longitudinally spaced needle ends, the first needle end having a needle point for piercing through the adjacent body tissues;
a suture filament having first and second longitudinally spaced filament ends, the first filament end attached to the second needle end;
a filament sheath having first and second sheath ends and defining a tubular sheath void, at least a portion of the suture filament enclosable within the sheath void, the first sheath end attached to the second needle end; and
an adhesive source, connectable to the second filament end and the second sheath end, the adhesive source being adapted to selectively provide an adhesive to the sheath void.

2. The apparatus of claim 1, wherein the adhesive source is adapted to selectively provide an adhesive to the suture filament.

3. The apparatus of claim 1, wherein the adhesive source is a breakable ampule contained within a flexible cartridge and connected to the second filament end.

4. The apparatus of claim 1, wherein the adhesive source is a variable volume reservoir selectively connectable to the second filament end.

5. The apparatus of claim 1, wherein the filament sheath includes at least one frangible area adapted to allow separation of the sheath into a plurality of sheath fragments.

6. A method of joining adjacent body tissues using adhesive sutures, the method comprising the steps of:
providing a suture apparatus including a suture needle having first and second longitudinally spaced needle ends, the first needle end having a needle point, and a suture filament having first and second longitudinally spaced filament ends, the first filament end attached to the second needle end;
providing a filament sheath having first and second sheath ends and defining a tubular sheath void, at least a portion of the suture filament enclosed within the sheath void, the first sheath end adjacent the second needle end;
approximating first and second body tissues into a suture position with at least a portion of the suture apparatus extending therethrough;
providing adhesive to at least a portion of the suture filament;
retaining at least a portion of the suture filament within the first and second body tissues; and
withdrawing the filament sheath from the first and second body tissues.

7. The method of claim 6, wherein the step of approximating first and second body tissues into a suture position with at least a portion of the suture apparatus extending therethrough includes the steps of:
penetrating a first side of the first body tissue with the first needle end;
drawing the suture needle and a portion of the suture filament through the first body tissue and out through a second side of the first body tissue spaced apart from the first side of the first body tissue;
retaining the second filament end and the second sheath end at a retention location adjacent the first side of the first body tissue;
penetrating a first side of a second body tissue with the first needle end;
drawing the suture needle and a portion of the suture filament through the second body tissue and out through a second side of the second body tissue spaced apart from the first side of the second body tissue;
drawing at least a portion of the filament sheath through the first and second body tissues concurrently with drawing the suture needle and a portion of the suture filament through the first and second body tissues; and
tensioning the suture filament to bring the second side of the first body tissue and the first side of the second body tissue closer together into a suture position.

8. The method of claim 6, wherein the step of providing adhesive to at least a portion of the suture filament includes the steps of:
providing an adhesive source connectable to the second sheath end;
approximating first and second body tissues into a suture position with at least a portion of the suture apparatus extending therethrough;
releasing adhesive from the adhesive source into the sheath void; and
directing adhesive to at least a portion of the suture filament through the sheath void.

9. The method of claim 8, wherein the step of releasing adhesive from the adhesive source into the sheath void includes the steps of:
providing an adhesive reservoir connected to the second sheath end, and
breaking an ampule within the adhesive reservoir.

10. The method of claim 8, wherein the step of releasing adhesive from the adhesive source into the sheath void includes the steps of:
selectively connecting a variable volume reservoir to the second sheath end, and
reducing the volume of the variable volume reservoir.

11. The method of claim 8, including the step of severing the suture filament adjacent the first side of the first body tissue and adjacent the second side of the second body tissue after the adhesive has been directed to at least a portion of the suture filament.
